(19) United States
(12) Patent Application Publication
(10) Pub. No.: US 2017/0065161 A1
(43) Pub. Date: Mar. 9, 2017

(54) Resolution Control Method for an Adaptive Optics System

(71) Applicant: CANON KABUSHIKI KAISHA, Tokyo (JP)

(72) Inventor: Koji Nozato, Rochester, NY (US)

(21) Appl. No.: 14/844,946

(22) Filed: Sep. 3, 2015

(51) Int. Cl. A61B 3/00; A61B 3/12; A61B 3/10

(52) U.S. Cl. CPC A61B 3/0008; A61B 3/1015; A61B 3/12

(57) ABSTRACT

An imaging apparatus with a wavefront controller and a method for controlling the apparatus. Sending control data based on a first setting to the wavefront controller to adjust an irradiation wavefront so as to set a beam shape of the irradiation beam of light. Receiving data that is representative of a wavefront of measurement light. Sending updated control data to the wavefront controller to readjust the irradiation wavefront based on the measurement wavefront data while at the same time also using the wavefront controller to control the beam shape of the irradiation beam of light based on the first setting.

### BACKGROUND

Field of Art

The present disclosure relates to a system and method for controlling the resolution in an ophthalmoscope.

Description of the Related Art

In recent years, scanning light ophthalmoscopes (SLOs) that irradiate the fundus with laser light in two dimensions and receive reflected light therefrom and an imaging apparatuses that utilizes the interference of low coherence light, such as an optical coherence tomographs (OCTs) have been developed as ophthalmic image pickup apparatuses. Thus, SLOs and OCTs have become important tools for the study of the human retina in both normal and diseased eyes.

The resolution of such SLOs have been improved by, for example, achieving high NA of irradiation laser light. However, when an image of the fundus is to be acquired, the image must be acquired through optical tissues including the cornea and the crystalline lens. As the resolution increases, the aberrations of the cornea and the crystalline lens have come to significantly affect the quality of acquired images.

The use of adaptive optics (AO) in AO-SLO and AO-OCT in which the AO is an optical correction system that both measures the aberration of the eye and corrects the aberration of the eye have been incorporated into some optical measurement systems. The AO-SLO and/or AO-OCT generally measure the wavefront of the eye using a Shack-Hartmann wavefront sensor system. A deformable mirror or one or more spatial-phase modulator(s) are driven to correct the measured wavefront. After which an image of the fundus is acquired, thus allowing the AO-SLO and/or AO-OCT to acquire high-resolution images.

Control of the AO system is done via a feedback loop system. The step of measuring aberrations and correcting the measured aberrations are processed one after another continuously. The AO performance is influenced by the condition of the eyes which consists of aberration, cataract, eye lid, eyelash, and the physical pupil size.

A typical AO-SLO uses a 6-8 mm diameter measurement beam. The optical resolution of the AO-SLO is governed by the diameter of the measurement beam. Having a 6-8 mm diameter measurement beam allows for the typical AO-SLO to resolve cells at the center of the fovea. But the larger the measurement beam the more the measurement is influenced by eye's aberration, cataract, eye lid, eyelash, and the physical pupil size. Thus, it can be difficult to take images from some subjects when a large diameter beam is used.

For this reason, it is useful for a measurement device to have the ability to change the diameter of the incident light (measurement beam) according to the imaging condition depending on the individual being measured and the amount of time available to make the measurement. While it is easier to achieve high resolution with a larger beam size, it is sometimes also easier to obtain an image with a smaller beam size.

Prior art methods of adjusting the beams size have used a pair of lenses or their equivalent and adjusted the distance between the two lenses. But beam diameter adjustment functionality as implemented in the prior art can be difficult to implement efficiently when the precision and stability requirements are high. This is because moving optical elements (such as optical lenses or curved mirrors) affect the precision, stability, and increase the cost of the entire system. In addition, the range of beam diameter adjustment is limited and fixed by the optical design and there is little flexibility, so the beam diameter cannot be changed according to subjects' condition in incremental steps.

What is needed is a flexible method of adjusting the beam spot size without affecting the cost, precision, or the stability of the optical system.

### SUMMARY

An adaptive optics fundus imaging apparatus with a wavefront controller system, and a method for controlling the adaptive optics fundus imaging apparatus that irradiates a subject's fundus with an irradiation light, and collects a measurement light from the subject. The method may comprise receiving a first setting. The method may also comprise sending control data based on the first setting to the wavefront controller to adjust an irradiation wavefront so as to set a beam shape of the irradiation beam of light. The method may also comprise receiving measurement wavefront data that is representative of a wavefront of the measurement light collected from the subject. The method may also comprise sending updated control data to the wavefront controller to readjust the irradiation wavefront based on the measurement wavefront data while at the same time also using the wavefront controller to control the beam shape of the irradiation beam of light based on the first setting.

An embodiment may also comprise receiving image data that may be representative of the collected measurement light, and may include constructing an image of the subject's fundus based upon the image data.

In an embodiment the first setting may be a resolution setting input by a user of the adaptive optics fundus imaging apparatus.

An embodiment may also comprise receiving a second setting different from the first setting. An embodiment may also comprise sending updated control data to the wavefront controller to readjust the irradiation wavefront based on the measurement wavefront data while at the same time also using the wavefront controller to change the beam shape of the irradiation beam of light based upon the second setting.

An embodiment may also comprise changing a size of an area of the subject's fundus that is imaged by the adaptive optics fundus imaging apparatus based upon the second setting.

In an embodiment, the measurement wavefront data may be updated periodically. In an embodiment, the control data may be updated periodically based upon the updated measurement wavefront data.

In an embodiment the wavefront controller may set the beam shape of the irradiation beam of light by blocking the light outside of the set beam shape.

In an embodiment the wavefront controller may include one or more spatial phase modulators.

In an embodiment, the first portion of the wavefront controller may be that portion of a control area of the wavefront controller that is illuminated by the irradiation beam of light which is outside of the beam shape. In an embodiment, the wavefront controller may set the beam shape of the irradiation beam of light by applying a periodic spatial phase modulation pattern to the first portion of the wavefront controller. In an embodiment, a period of the periodic spatial phase modulation pattern is substantially equal to half a wavelength of the irradiation light.

In an embodiment, the periodic spatial phase modulation pattern may be periodic along a first axis and is substantially constant along a second axis which is substantially orthogonal to the first axis.

In an embodiment, the periodic spatial phase modulation pattern may be substantially radially symmetric.

In an embodiment, the beam shape may be radially symmetric and a radial axis of symmetry for the periodic spatial phase modulation pattern may be substantially coaxial with a center of the beam shape of the irradiation beam of light.

In an embodiment, the wavelength of the irradiation light may be selected from the group consisting of: a peak wavelength of the irradiation light; a mean wavelength of the irradiation light; and a median wavelength of the irradiation light.

In an embodiment, a first portion of the wavefront controller may be that portion of a control area of the wavefront controller that is illuminated by the irradiation beam of light which is outside of the beam shape. The wavefront controller may sets the beam shape of the irradiation beam of light by applying a spatial phase modulation pattern to the first portion of the wavefront controller which causes a first aberration to be added to the irradiation beam of light that illuminates the first portion of the wavefront controller.

In an embodiment, the first may aberration includes one or more of: a defocus aberration; a conic aberration; one directional tilt aberration; a two directional tilt aberration; and a radial tilt aberration.

In an embodiment, the measurement wavefront data may include information about aberrations introduced to the measurement light by the subject.

In an embodiment the beam shape may be a circle, the wavefront controller may be used to set a beam diameter of the irradiation light.

In an embodiment, the first setting may be a resolution setting and a size of the beam shape may be calculated based on the resolution setting.

An embodiment may be a non-transitory computer readable medium may be encoded with instructions for one or more processors to control the adaptive optics fundus imaging apparatus with the wavefront controller that irradiates a subject's fundus with an irradiation light, and collects a measurement light from the subject.

An embodiment may be an adaptive optics fundus imaging apparatus. The embodiment may comprise a wavefront controller, that changes a wavefront of irradiation light that is used to irradiate a subject's fundus. The embodiment may comprise a wavefront sensor for measuring a wavefront of measurement light collected from the subjects fundus that outputs measurement wavefront data that is representative of the wavefront of the measurement light. The embodiment may comprise one or more processors. In the embodiment, the one or more processors may receive a first setting. In the embodiment, the one or more processors may send control data based on the first setting to the wavefront controller to adjust an irradiation wavefront so as to set a beam shape of the irradiation beam of light. In the embodiment, the one or more processors may receive the measurement wavefront data from the wavefront sensor. In the embodiment, the one or more processors may send updated control data to the wavefront controller to readjust the irradiation wavefront based on the measurement wavefront data while at the same time also using the wavefront controller to control the beam shape of the irradiation beam of light based on the first setting.

### BRIEF DESCRIPTION OF THE DRAWINGS

The accompanying drawings, which are incorporated in and constitute a part of the specification, illustrate exemplary embodiments.

FIGS. 1A-C are illustrations of an ophthalmoscope in which an embodiment may be implemented.

FIG. 2 is an illustration of a process that may be used in an embodiment.

FIG. 3 is an illustration of how the areas of a phase map may be divided in an embodiment.

FIGS. 4A-H are illustrations of modulation patterns that may be used in an embodiment.

FIGS. 5A-D are illustrations of phase maps that may be used in an embodiment.

FIGS. 6A-B are illustrations of images that may be produce by an embodiment.

FIG. 7 is an illustration of a PC and a controller that may be used in an embodiment.

### DESCRIPTION OF THE EMBODIMENTS

Embodiments will be described below with reference to the attached drawings. Like numbers refer to like elements throughout. Exemplary embodiments will be described in detail with reference to the drawings below. It shall be noted that the following description is merely illustrative and exemplary in nature, and is in no way intended to limit the disclosure and its applications or uses. The relative arrangement of components and steps, numerical expressions and numerical values set forth in the embodiments do not limit the scope of the disclosure unless it is otherwise specifically stated. Techniques, methods, and devices which are well known by individuals skilled in the art may not have been discussed in detail since an individual skilled in the art would not need to know these details to enable the embodiments discussed below. Further, an image photographing apparatus as disclosed in the following which is used inspect an eye as described below may also be used to inspect other objects including but not limited to skin, and internal organs.

### Ophthalmoscope

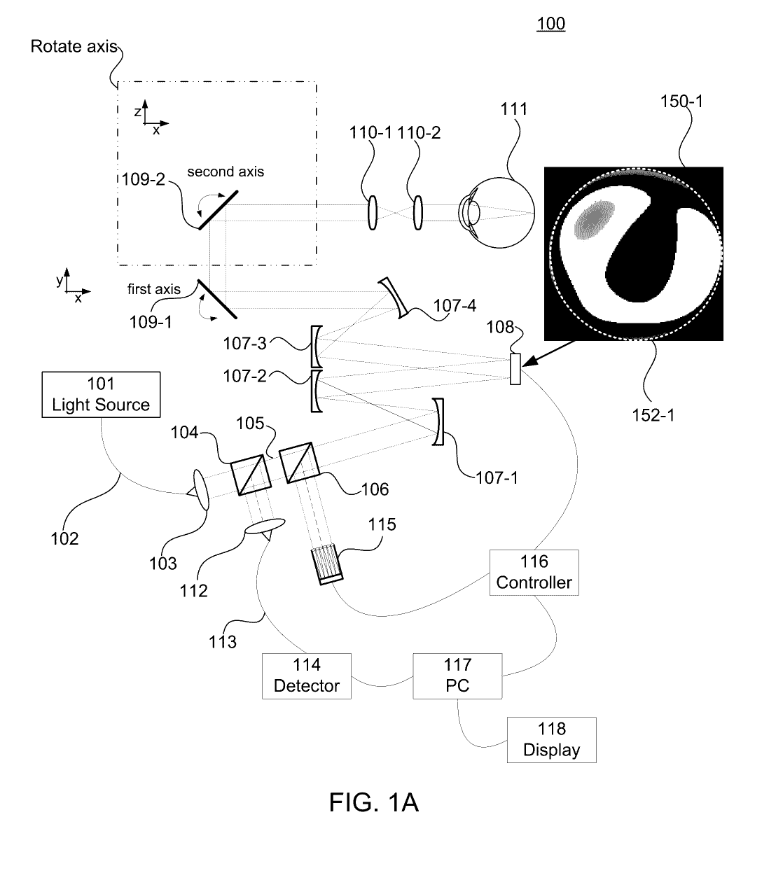

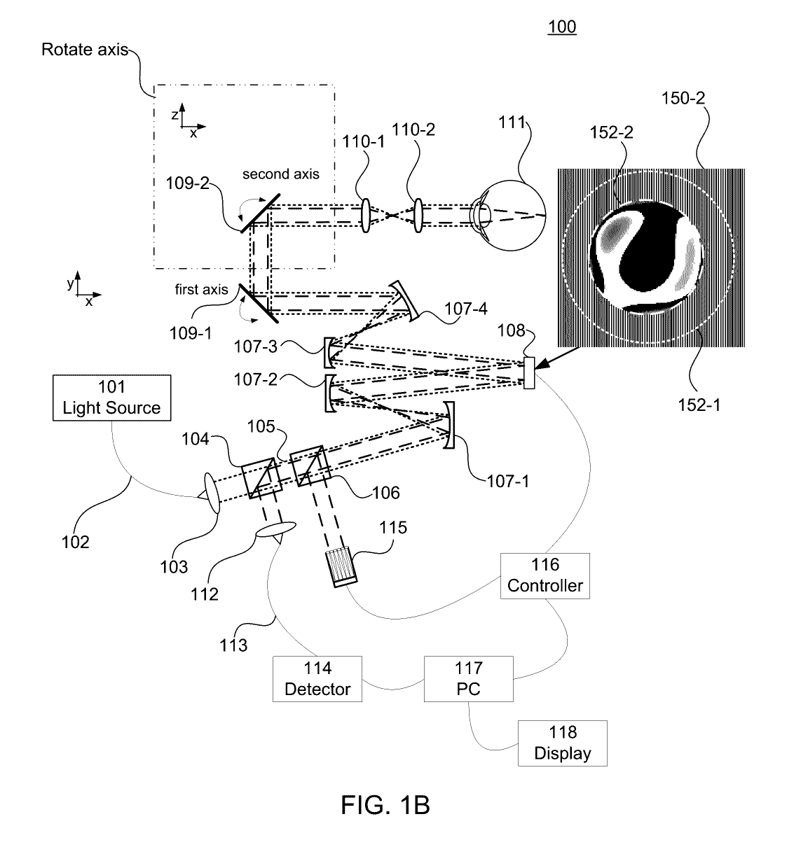

A first embodiment is described with reference to a fundus image photographing apparatus (ophthalmoscope) such as the photographing apparatus illustrated in FIG. 1A.

Embodiments are directed towards systems, methods, non-transitory computer readable medium, and software which are used in connection with an imaging system such as an ophthalmoscope 100. FIG. 1A is an illustration of an exemplary ophthalmoscope 100. An ophthalmoscope 100 is a system or apparatus for obtaining information about an interior portion of the eye 111 (e.g., the fundus).

An exemplary embodiment may be a scanning ophthalmoscope. A scanning ophthalmoscope scans a spot across the eye 111. The spot may be a spot of light from a light source 101 that is scanned across the eye 111.

In an exemplary embodiment 100, the spot of light is produced by a light source 101. The light source 101 may be incorporated into the ophthalmoscope 100; alternatively, the ophthalmoscope 100 may include an input for receiving the light source 101. The input for the light source 101 may be a fiber optic input or a free space input. The light source 101 may be a laser, a broadband light source, or multiple light sources. In an exemplary embodiment, the light source 101 is a super luminescent diode (SLD) light source having a wavelength of 840 nm. The wavelength of the light source 101 is not particularly limited, but the wavelength of the light source 101 for fundus image photographing is suitably set in a range of approximately 800 nm to 1,500 nm in order to reduce glare perceived by a person being inspected and to maintain imaging resolution.

In an exemplary embodiment, light emitted from the light source 101 passes through a single-mode optical fiber 102, and is radiated as collimated light (measuring light 105) by a collimator 103.

In exemplary an embodiment, the polarization of the irradiated light may be adjusted by a polarization adjusting member 119 (not shown) provided in a path of the single-mode optical fiber 102. In an alternative configuration, the light source 101 is polarized and single-mode optical fiber 102 is polarization maintain fiber. In another configuration, the polarization adjusting member may be placed after the collimator 103. Alternatively, the polarization adjusting member may be replaced with a polarizer.

The measuring light 105 radiated from the collimator 103 passes through a light division portion 104 including a beam splitter. An exemplary embodiment includes an adaptive optical system.

The adaptive optical system may include a light division portion 106, a wavefront sensor 115, wavefront adjustment device 108, and reflective mirrors 107-1 to 107-4 for guiding the measuring light 105 to and from those components. The reflective mirrors 107-1 to 107-4 are provided to guide the measuring light 105 to and from the pupil of an eye 111, the wavefront sensor 115, and the wavefront adjustment device 108. The reflective mirrors may be replaced with suitable optics, such as lenses and/or apertures. The wavefront sensor 115 and the wavefront adjustment device 108 may be in an optically conjugate relationship. A beam splitter may be used as the light division portion 106. The wavefront sensor 115 may be a Shack-Hartmann sensor or other type of sensor that gathers information that is representative of the wavefront of light coming from the subject.

The measuring light 105 passing through the light division portion 106 is reflected by the reflective mirrors 107-1 and 107-2 so as to enter the wavefront adjustment device 108. The measuring light 105 is reflected by the wavefront adjustment device 108 and is further reflected by the reflective mirrors 107-3 and 107-4.

The wavefront adjustment device 108 maybe a transmissive device or a reflective device. The wavefront adjustment device 108, is an addressable spatial light phase modulator that allows relative phases across a beam coming into the wavefront adjustment device 108 to be adjusted such that relative phases across the beam coming out of the wavefront adjustment device 108 are adjustable. In an exemplary embodiment, one or two spatial phase modulators including a liquid crystal element is used as the wavefront adjustment device 108. The liquid crystal element may modulate a phase of only a specific polarized component. In which case, two liquid crystal elements may be employed to modulate substantially orthogonal polarized components of the measuring light 105. In an alternative embodiment, the wavefront adjustment device 108 is a deformable mirror.

The measuring light 105 reflected off mirror 107-4 is two-dimensionally scanned by a scanning optical system 109. In an exemplary embodiment, the scanning optical system 109 includes a first scanner 109-1 and a second scanner 109-2. The first scanner 109-1 rotates around the first axis, while the second scanner 109-2 rotates around a second axis. The first axis is substantially orthogonal to the second axis. Substantially in the context of the present disclosure means within the alignment and measurement tolerances of the system.

FIG. 1A illustrates the first scanner 109-1 rotating in the x-y plane, while the second scanner 109-2 is rotating in the z-x plane. In the context of the present disclosure, rotating the measuring light 105 in a first plane around the first axis is equivalent to rotating the measuring light 105 in the first plane and is equivalent to scanning the spot of light in the main scanning direction or the lateral direction of the object being imaged. In the context of the present disclosure, rotating the measuring light 105 in a second plane around the second axis is equivalent to rotating the measuring light 105 in the second plane and is equivalent to scanning the spot of light in the sub-scanning direction or the longitudinal direction of the object being imaged. The sub-scanning direction is substantially orthogonal to the main scanning direction.

A scanning period of the first scanner 109-1 is less than the scanning period of the second scanner 109-2. The order of the first scanner 109-1 and the second scanner 109-2 may be exchanged without impacting the operation of an exemplary embodiment. The first scanner 109-1 may operate in a resonant scanning mode.

In an exemplary embodiment, the scanning optical system 109 may be a single tip-tilt mirror that is rotated around the first axis and around the second axis that is substantially orthogonal to the first axis. An exemplary embodiment may also use non-mechanical beam steering techniques.

In an exemplary embodiment, the first scanner 109-1 and the second scanner 109-2 are galvano-scanners. In another exemplary embodiment, one of the first scanner 109-1 and the second scanner 109-2 is a resonant scanner. The resonant scanner may be used for the main scanning direction. The resonant scanner may be tuned to oscillate at a specific frequency. There may be additional optical components, such as lenses, mirrors, apertures, etc. between the scanners 109-1, 109-2, and other optical components, these may be arranged such that the light is focused onto the scanners, in a manner that is optically conjugate with all of or one or more of the subject 111, the wavefront adjustment device 108, the wavefront sensor 115, and a detector 114

The measuring light 105 scanned by the scanning optical system 109 may be radiated to the eye 111 through eyepieces 110-1 and 110-2. The measuring light radiated to the eye 111 is reflected, scattered, or absorbed on the fundus. When the eyepieces 110-1 and 110-2 are adjusted in position, suitable irradiation may be performed in accordance with the diopter of the eye 111. Lenses may be used for the eyepiece portion in this embodiment, but other optical components such as spherical mirrors may also be used.

Light which is produced by reflection, fluorescence, or scattering on a retina of the eye 111 then travels in the reverse direction along the same path as in the case of incident light. A part of the reflected light is reflected by the light division portion 106 to the wavefront sensor 115 to be used for measuring a light beam wavefront.

In an exemplary embodiment, a Shack-Hartmann sensor is used as the wavefront sensor 115. However, an exemplary embodiment is not limited to a Shack-Hartmann sensor. Another wavefront measurement unit, for example, a curvature sensor may be employed or a method of obtaining the wavefront by reverse calculation from the formed spot images may also be employed.

In FIG. 1A, when the reflected light passes through the light division portion 106, a part thereof is reflected on the light division portion 104 and is guided to a light intensity sensor 114 through a collimator 112 and an optical fiber 113. The light intensity sensor 114 converts the light into an electrical signal. The electrical signal is processed by a control unit 117 into an image of the object, and the image is displayed on a display 118.

The wavefront sensor 115 is connected to an adaptive optics control unit 116. The received wavefront is transferred to the adaptive optics control unit 116. The wavefront adjustment device 108 is also connected to the adaptive optics control unit 116 and performs modulation as instructed by the adaptive optics control unit 116. The adaptive optics control unit 116 calculates a modulation amount (correction amount) for correction to obtain a wavefront having less aberration based on the wavefront obtained by a measuring result of the wavefront sensor 115, and instructs the wavefront adjustment device 108 to perform the modulation according to the modulation amount. The wavefront measurement and the instruction to the wavefront adjustment device are repeated and feedback control is performed so as to obtain a suitable wavefront.

In an exemplary embodiment the light division portions 104 and/or 106 are fused fiber couplers. In an alternative exemplary embodiment, the light division portions 104 and/or 106 may include partially reflective mirrors. In another alternative exemplary embodiment, the light division portions 104 and/or 106 may include dichroic reflectors, in which case a different wavelength of light is used for detecting the phase than is used for detecting the image.

The detector 114 may detect reflections or fluorescence associated with the scanning spot. The detection system may make use confocal microscopy techniques in which an aperture associated with the scanning spot is used to increase the resolution and/or contrast of the detection system.

### Adaptive Optics

The adaptive optics system described above includes at least the wavefront sensor 115 and the wavefront adjustment device 108 so that the aberration of the subject's eyes can be measured and compensated for. A deformable mirror (DM) or a spatial light phase modulator (SLM) can be used as the wavefront adjustment device 108. Since the typical SLM has a larger number of actuators than a typical DM, it can modulate the wavefront more precisely than the DM can. The control area of the wavefront adjustment device 108 corresponds to the beam size 152-1 of the incident light. A first phase map 150-1 on the wavefront adjustment device 115 corresponds to the beam size 152-1. FIG. 1A includes an illustration of a gray scale image of the phase adjustment that is represented as the first phase map 150-1 as applied to the wavefront adjustment area on the wavefront adjustment device 108 based on measurements made by the wavefront sensor 115. The white dashed line 152-1 is an illustration of the beam size at the wavefront adjustment device 108. The black portion of the first phase map 150-1 represents the regions with zero phase adjustment while the brighter portions represent the area with larger phase adjustments.

In an exemplary embodiment, the wavefront adjustment device 108 is used for blocking a portion of a large size beam so as to make the beam diameter smaller as illustrated in FIG. 1B. A liquid crystal on silicon spatial light modulator (LCOS-SLM) may be used as the wavefront adjustment device 108. The LCOS-SLM 108 can be controlled to provide a precise spatial modulation of the phase of the beam that is used to illuminate the subject. This precise spatial modulation of the phase of the beam also controls the sensing area of the wavefront sensor according to changes in the beam diameter.

FIG. 1B is an illustration of an embodiment also illustrated in FIG. 1A except for the controller 116 uses a second phase map 150-2 to control the wavefront adjustment device 108. The second phase map 150-2 is illustrated with two circles. A first circle 152-1 with short dashed lines and a second circle 152-2 with longer dashed lines. The first circle 152-1 represents a beam with a larger beam diameter as shown by the beam made up of short dashed lines that are passed along by the various optical components. The second circle 152-2 represents a beam with a smaller beam diameter as shown by the beam made up of long dashed lines that are passed along by the various optical components. FIG. 1B also shows how a portion of the larger beam may be blocked by the iris of the eye. The second phase map 150-2 illustrates how the control area is divided into two areas. A first area inside the second circle 152-2 is controlled based on information from the wavefront sensor 115. While a second area outside of the second circle 152-2, has a pattern which causes the light not to be focused onto the retina of the subject.

FIG. 1C is an illustration of the embodiment also illustrated in FIGS. 1A-B in which the controller 116 uses the second phase map 150-2 to control the wavefront adjustment device 108 is the same as in FIG. 1B. FIG. 1C illustrates how the larger beam does not travel along with the smaller beam after the wavefront adjustment device 108.

### Process

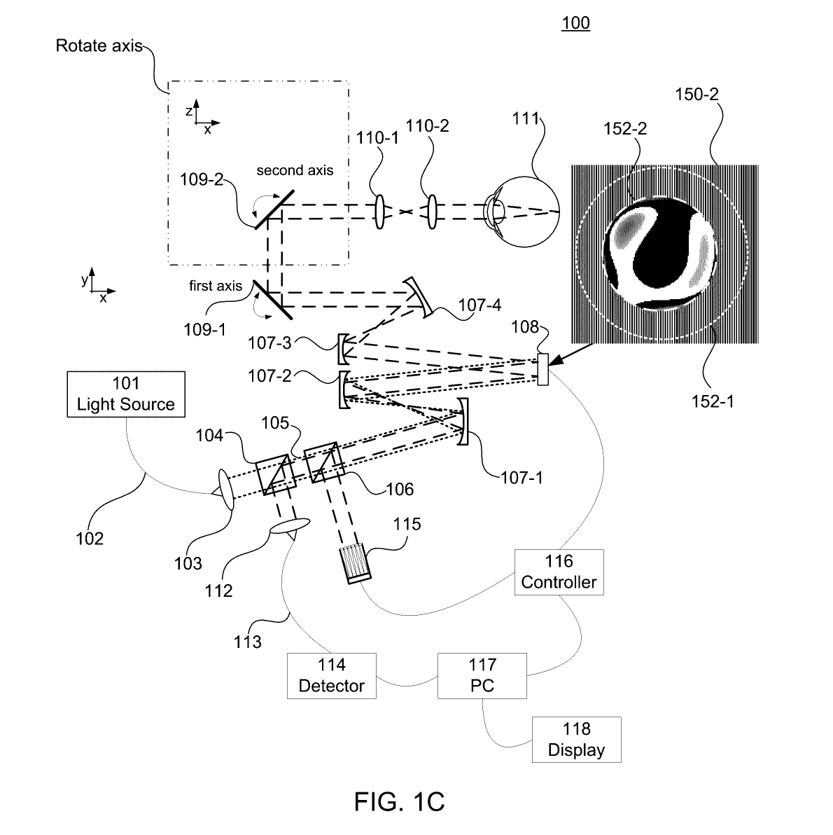

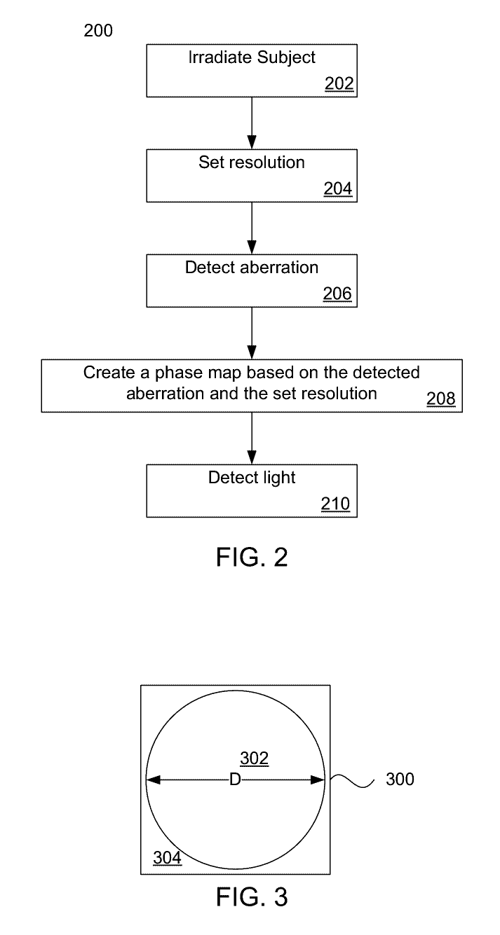

FIG. 2 is an illustration of a method 200 that may be used in an embodiment. The method 200 may include a first step 202 of irradiating a light beam from a light source 101 onto a subject 111. In a second step 204, the resolution is set. This may be set by an operator or may be set based upon information that is known about the subject 111 being imaged. For example, information about the subject may include the subject's prescription, the subject's cataract condition, or other geometrical information about the subject 111.

In a third step 206 aberration is detected by the wavefront sensor 115. The aberration may be due to aberrations introduced by the subject 111. For example, the crystalline lens of the eye may introduce aberrations. There may also be a baseline aberration due to the optical design of the embodiment. This baseline aberration may be constant or may be affected by environmental variables such as temperature and/or humidity. The controller 116 may read the information from the wavefront sensor 115.

In a fourth step 208, a wavefront of the irradiation beam is adjusted by the wavefront adjustment device 108. The controller 116 uses information from the wavefront sensor 115 to create a phase map 150 which is then used to control the wavefront adjustment device 108. The controller 116 may communicate with a PC 117 when producing the phase map 150. The phase map 150 is modified based on the set resolution. A beam diameter D is set based upon the set resolution. The beam diameter D may be set according to equation (1) below:

$$D = \frac{4 λ}{π} \frac{f}{resolution} \qquad ( 1 )$$

In which: λ is the wavelength of the illumination light (ex.: 760-840 nm); f is the focal length of the eye (ex. 17 mm); and resolution is the spot size at the eye. FIG. 3 is an illustration of how the area 300 of the phase map 150 is divided into two areas 302 and 304. A first area 302 is the area inside a circle with the beam diameter D. The second area 304 is the area outside the circle with the beam diameter D. The first area 302 of the phase map is adjusted as part of a feedback loop that changes the first area 302 based on information from the wavefront sensor 115 and the previous setting in the first area 302. The second area 304 of the phase map has a pattern that causes the illumination light to be blocked. In an alternative embodiment, one or more fixed apertures may be placed at focal points along the optical path such that light that falls on the second area 304 of the phase map is blocked due to the path change caused by the pattern in the second are 304.

In a fifth step 210, the measurement light is detected by a detector 114 which are used to construct images of the fundus.

Steps 202-204 may operate continuously and independently. The process may include constantly checking to see if there are changes to the resolution setting in step 204. If there are changes to the resolution setting then the phase map is changed accordingly. Step 204 may be measured independently of step 206. As soon as changes in the aberrations are detected then the phase map is changed. The step 208, may make changes as soon changes in the aberrations and/or resolution setting is detected.

### Modulation Patterns

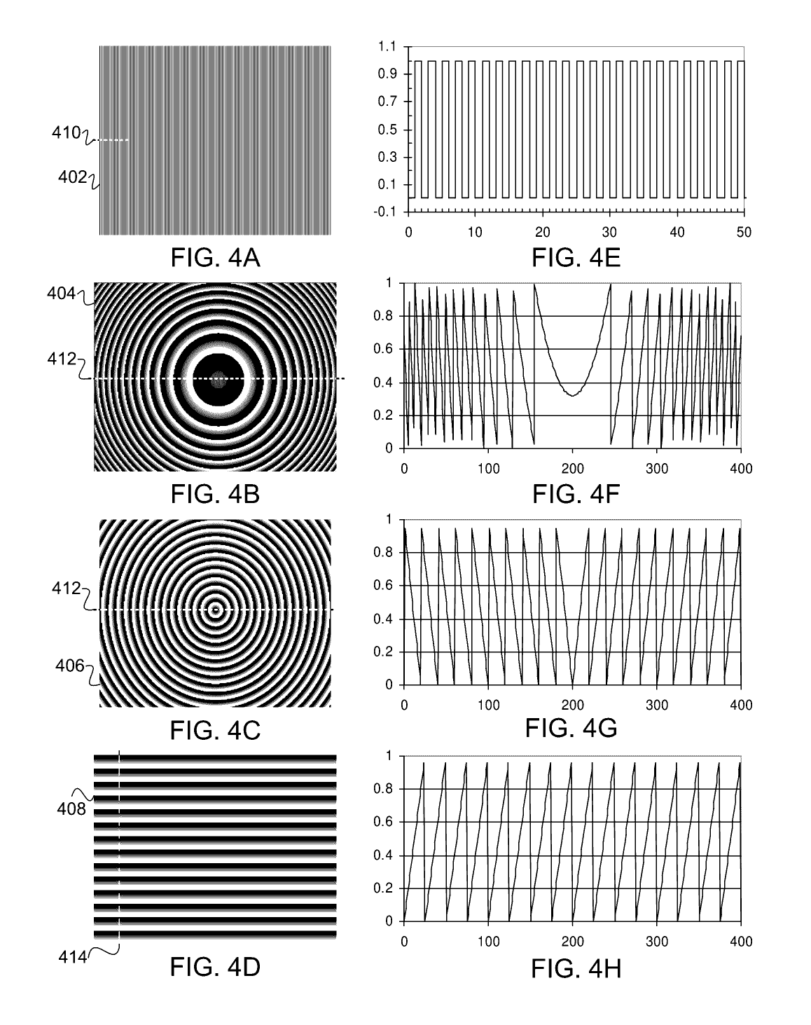

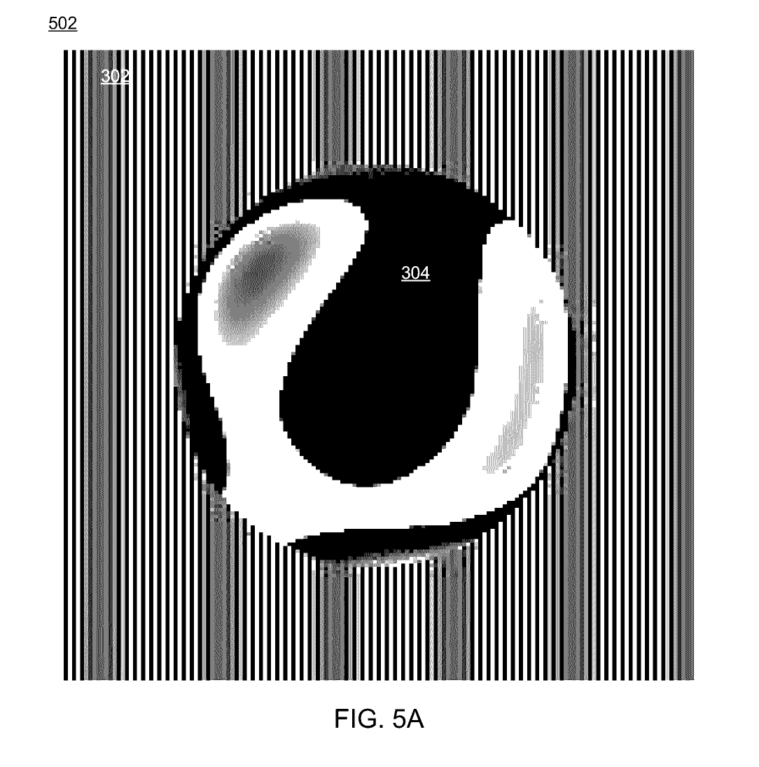

Several different types of patterns can be used in the second area 304 to block illumination light. The following patterns may be stored in a database or may be calculated on the fly based upon parameters entered by a user, parameters taken from a database, these parameters may be specified in terms of relative pixel positions, or specified in specific units such as micrometers. FIG. 4A is an illustration of a stripe pattern 402 that can be used in the second area 304 to block illumination light by diffraction. The stripe pattern 402 is periodic and may have a period of around 100 μm. The modulation of the stripe pattern 402 may be a binary modulation, a triangle wave modulation, a sawtooth wave modulation, or a sinusoidal wave modulation. FIG. 4E is an illustration of the normalized phase of an exemplary stripe pattern 402 with binary modulation along the white line 410 illustrated in FIG. 4A as a function of the pixel count of the wavefront adjustment device 108. A representative portion of the pattern 402 is shown for clarity. The orientation may be along one of scanning axes or at an angle with it. The stripe pattern will cause the light to spread due to diffraction.

Another type of pattern that can be used in the second area 304 is a defocus pattern 404 illustrated in FIG. 4B. The light that lands on the second area 304 will go outside of the optical system 100 or it will get blurred on the subject 111 and the light detector 114. The defocus pattern 404 may have pattern such as φ=f(ρ). In which ρ is the normalized radius and φ is the normalized phase. One example of the modulation pattern of the LCOS-SLM is described by equation (2) below:

$$φ = f ( ρ ) = \frac{\{ A \times ( - 1 + 2 {ρ}^{2} ) \} mod λ}{λ} \qquad ( 2 )$$

In which A is a scaling parameter associated with the amplitude of the defocus in μm and λ (μm) is the median wavelength of the light from the light source 101 or the incident light used for measuring the fundus. The light reflected on the area 304 is defocused by the pattern and it's easy to eliminate from the main beam. Other examples of defocus patterns 404 may be used which meet the goal of defocusing the light from the light source 101 or the incident light used for measuring the fundus. FIG. 4F is an illustration of the normalized phase φ as a function of the pixel count of the wavefront adjustment device 108 along the line 412 as illustrated in FIG. 4B. Note that in FIG. 4F the peaks and troughs of do not extend to the normalized limits of 0 and 1, this is due the finite number of pixels in the wavefront correction device 108. The discretization step in which phase adjustment is calculated for each pixel may be done based upon a center pixel value, an median value across the pixel, mean value across the pixel, maximum value across the pixel, or a minimum value across the pixel. The modulo calculation and the normalization division calculation may be performed after the discretization step.

Another type of pattern that can be used in the second area 304 is a conic pattern 406 illustrated in FIG. 4C. The light that lands on the second area 304 will go outside of the optical system 100 or it will get blurred on the subject 111 and the light detector 114. The conic pattern 406 may be a pattern such as φ=g(ρ). This conic pattern 406 works like an Axicon lens which reflects light radially without focusing. One example of the modulation pattern of the LCOS-SLM is described by equation (3) below:

$$φ = g ( ρ ) = \frac{\{ B \times ( - 1 + 2 ρ ) \} mod λ}{λ} \qquad ( 3 )$$

In which B is a scaling parameter associated with the amplitude of the radially spreading of the light in units of (μm). This pattern 404 causes the light to spread out radially such that the unused light can be easily blocked with one or more apertures along the main axis or by the inherent apertures associated with the intervening optical components between the fundus and the wavefront correction device 108. Other examples of conic patterns 406 may be used which meet the goal of spreading the light from the light source 101 or the incident light used for measuring the fundus. FIG. 4G is an illustration of the normalized phase φas a function of the pixel count of the wavefront adjustment device 108 along the line 412 as illustrated in FIG. 4C.

Another type of pattern that can be used in the second area 304 is a tilt pattern 408 illustrated in FIG. 4D. The light that lands on the second area 304 will go outside of the optical system 100 or it will get blurred on the subject 111 and the light detector 114. The tilt pattern 408 is a periodic modulation of the phase along one modulation axis which reflects the light outside of the main axis without focusing φ=h(y). One example of this modulation pattern of the LCOS-SLM is described by equation (4) below:

$$φ = h ( y ) = \frac{\{ C \times y \} mod λ}{λ} \qquad ( 4 )$$

h(y)={A×y} mod λ. In which C is a unitless scaling parameter associated with the amplitude of the tilt applied to the light and y is the coordinate in units of (μm) along the vertical direction. This tilt pattern 308 can be used in the case that the unused light may be blocked on specific side of the main axis. The alignment of the modulation axis may be independent of the scanning axes. The amount and direction of the tilt can be set according to other components of the optical system such that the light exits illumination and detection systems. The modulation of the tilt pattern 408 may be a binary modulation, a triangle wave modulation, a sawtooth wave modulation, or a sinusoidal wave modulation. Other examples of tilt patterns 408 may be used which meet the goal of spreading the light from the light source 101 or the incident light used for measuring the fundus in one direction. FIG. 4H is an illustration of the normalized phase φas a function of the pixel count of the wavefront adjustment device 108 along the line 414 as illustrated in FIG. 4D. Multiple modulation patterns may be combined together to meet the goals of dispersing the unwanted light.

### Modulation Pattern and Adaptive Optics

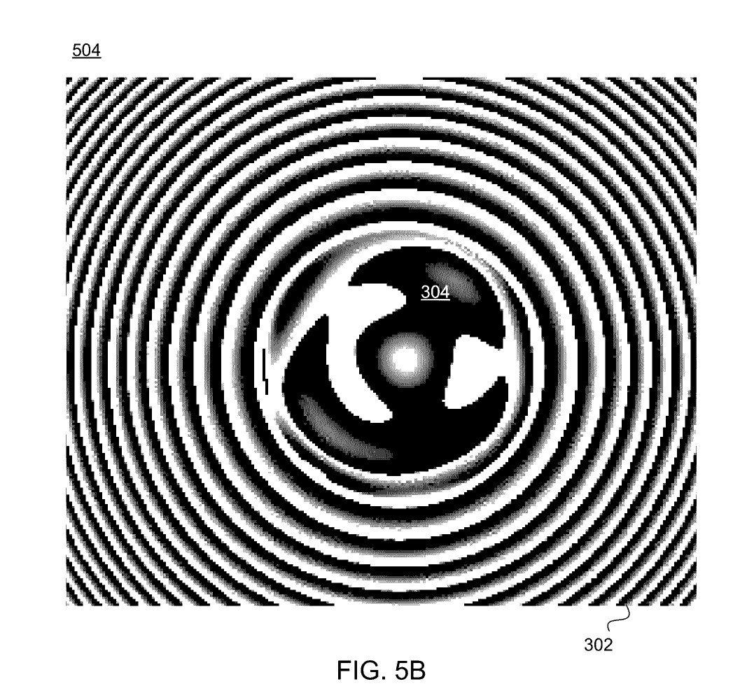

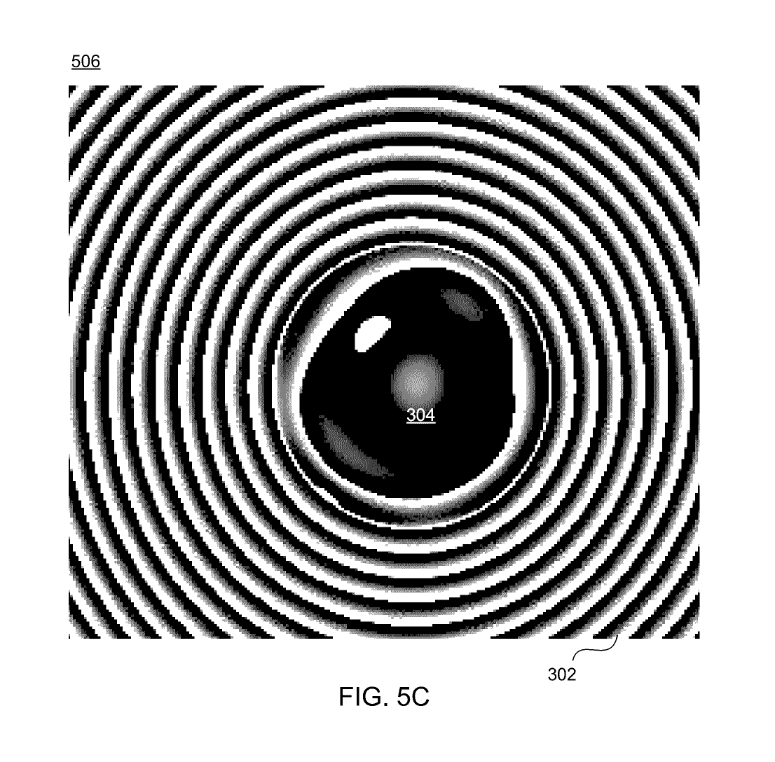

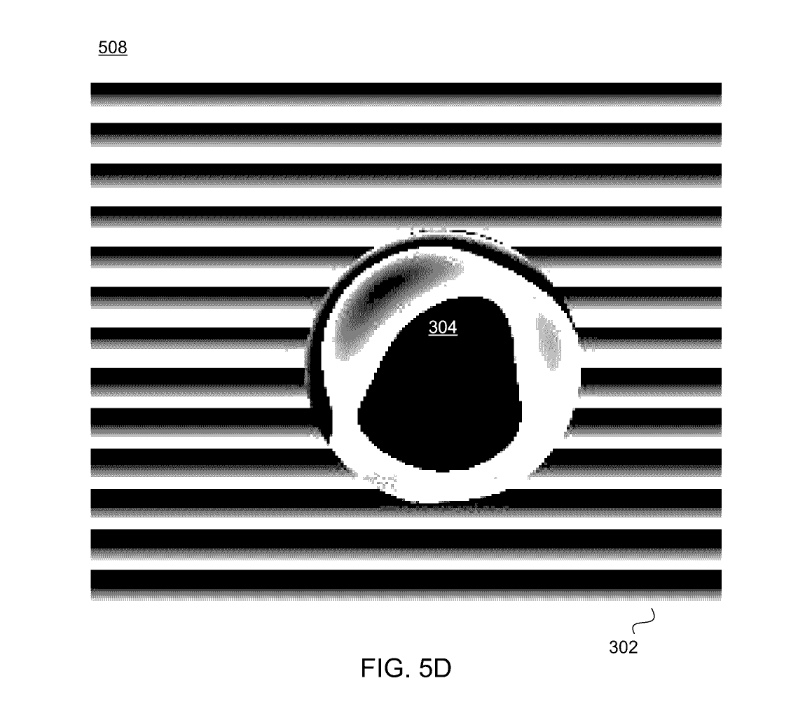

FIG. 5A is an illustration of a first resultant phase map 502 of the modulation pattern 402 in the second area 304 and an adaptive optics pattern in the first area 302.

FIG. 5A is an illustration of a first resultant phase map 502 of the stripe type modulation pattern 402 in the second area 304 and an adaptive optics pattern in the first area 302.

FIG. 5B is an illustration of a first resultant phase map 504 of the defocus type modulation pattern 404 in the second area 304 and an adaptive optics pattern in the first area 302.

FIG. 5C is an illustration of a first resultant phase map 506 of the conic type modulation pattern 406 in the second area 304 and an adaptive optics pattern in the first area 302.

FIG. 5D is an illustration of a first resultant phase map 508 of the tilt type modulation pattern 408 in the second area 304 and an adaptive optics pattern in the first area 302.

By using the phase maps such as the 502-508 illustrated in FIGS. 5A-D an adaptive optics imaging device such as an AO-SLO can have the added capability of changing the resolution without any additional hardware. It also allows provides the capability of changing the resolution dynamically and gradually without using moving parts. The applicant has found that when an LCOS is used as the wavefront adjustment device 108 it is possible to block up to 90% of the light by using an appropriate modulation pattern in the second area 304.

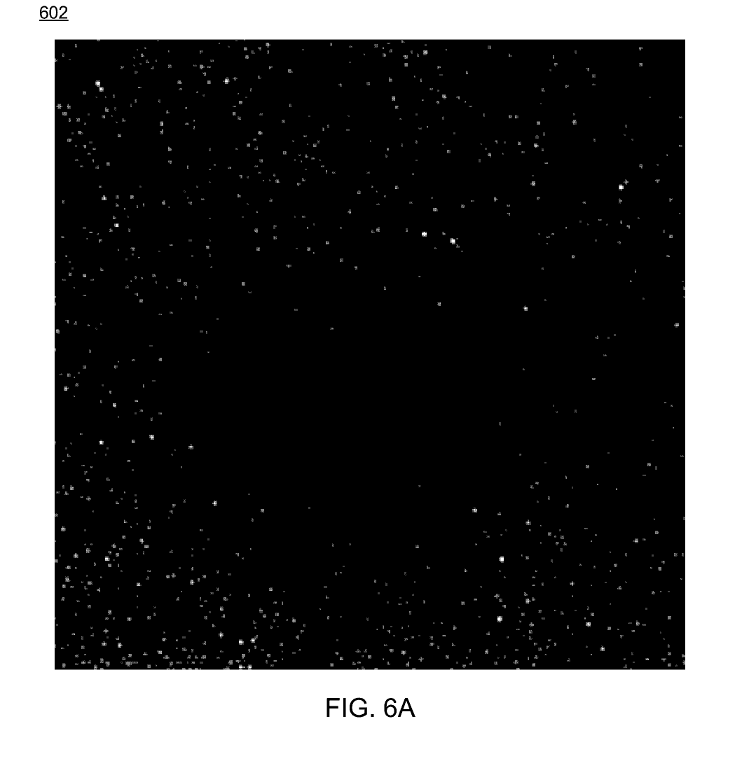

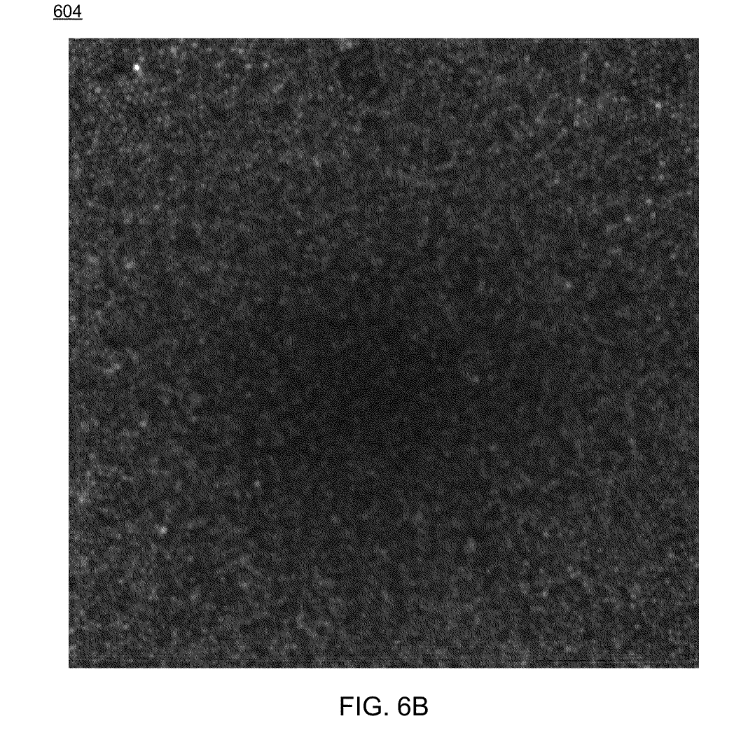

FIG. 6A is an illustration of a first image 602 obtained with the AO-SLO such as ophthalmoscope 100 in which the system has been set to a normal high resolution mode in which a typical phase map such as 150-1 is used. Cone photoreceptors near the center are resolved. Cone photoreceptors are smallest at the center and become larger gradually according to the distance from the center.

FIG. 6B is an illustration of a second image 604 obtained with the AO-SLO such as ophthalmoscope 100 in which the system has been set to a low resolution mode in which a typical phase map such as 150-2 or 502 is used. Cone photoreceptors near the center are not resolved. Only photoreceptors at about 100 μm or more from the center are resolved.

### Controller

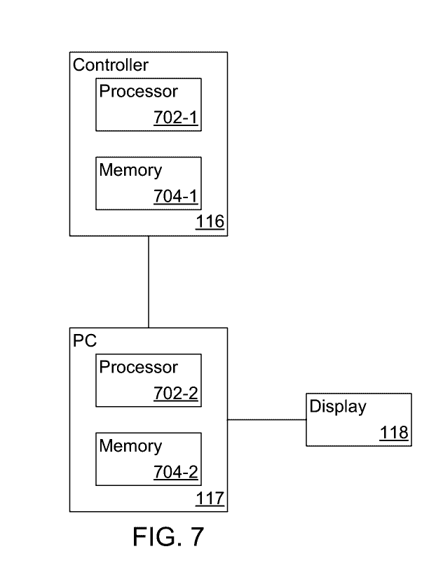

FIG. 7 is an illustration of the PC 116 and controller 117 that may be used in an embodiment. The controller 116 receives input signals and outputs control signals. The controller 116 may be a general purpose computer, a device specifically designed to controller the ophthalmoscope 100, or a hybrid device that uses some custom electronics along with a general purpose computer 117. The input signals and control signals maybe digital signals or analog signals. The controller 116 may include an analog to digital converter (ADC) and a digital to analog converter (DAC). The input signals may include one more signals such as a signal from the wavefront sensor 115, a signal from the detector 113, and one or more signals from one or more other sensors. The control signals may include a first control signal to a wavefront adjustment device 108 and signals to one or more of the scanners 109-1 and 109-2. The control signals may include additional signals to other components of the ophthalmoscope 100.

The controller 116 includes a processor 702-1. The processor 702-1 may be a microprocessor, a CPU, an ASIC, a DSP, and/or a FPGA. The processor 702-1 may refer to one or more processors that act together to obtain a desired result. The controller 116 may include a memory 704-1. The memory 704-1 may store calibration information. The memory 704-1 may also store software for controlling the ophthalmoscope 100. The memory 704 may be a form of a non-transitory computer readable storage medium. The non-transitory computer readable storage medium may include, for example, one or more of a hard disk, a random-access memory (RAM), a read only memory (ROM), a distributed storage system, an optical disk (CD, DVD or Blu-Ray Disc, a flash memory device, a memory card, or the like. The non-transitory computer readable storage medium may be accessed via a network connection.

The controller 116 may be connected to a computer (PC) 117 via a direct connection, a bus, or via a network. The computer 117 may include input devices such as a keyboard, a mouse, or a touch screen. The controller 116 may include input devices such as a keyboard, a mouse or a touch screen, knobs, switches, and/or buttons. The computer 117 may be connected to a display 118. The results of the ophthalmoscope 100 may be presented to a user via the display 118. The production of the phase maps which are used to control the wavefront adjustment device 108 may be created by the controller 116 independently of the PC 117 or with the help of the PC 117. The PC may include a processor 702-2 and a memory 704-2.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

What is claimed is:

1. A method for controlling an adaptive optics fundus imaging apparatus with a wavefront controller that irradiates a subject's fundus with an irradiation light, and collects a measurement light from the subject, comprising:
receiving a first setting;
sending control data based on the first setting to the wavefront controller to adjust an irradiation wavefront so as to set a beam shape of the irradiation beam of light;
receiving measurement wavefront data that is representative of a wavefront of the measurement light collected from the subject; and
sending updated control data to the wavefront controller to readjust the irradiation wavefront based on the measurement wavefront data while at the same time also using the wavefront controller to control the beam shape of the irradiation beam of light based on the first setting.

2. The method according to claim 1, further comprising receiving image data that is representative of the collected measurement light, and constructing an image of the subject's fundus based upon the image data.

3. The method according to claim 1, wherein the first setting is a resolution setting input by a user of the adaptive optics fundus imaging apparatus.

4. The method according to claim 1, further comprising:
receiving a second setting different from the first setting;
sending updated control data to the wavefront controller to readjust the irradiation wavefront based on the measurement wavefront data while at the same time also using the wavefront controller to change the beam shape of the irradiation beam of light based upon the second setting.

5. The method according to claim 4, further comprising:
changing a size of an area of the subject's fundus that is imaged by the adaptive optics fundus imaging apparatus based upon the second setting.

6. The method according to claim 1, wherein:
the measurement wavefront data is updated periodically; and
the control data is updated periodically based upon the updated measurement wavefront data.

7. The method according to claim 1, wherein the wavefront controller sets the beam shape of the irradiation beam of light by blocking the light outside of the set beam shape.

8. The method according to claim 1, wherein the wavefront controller includes one or more spatial phase modulators.

9. The method according to claim 1, wherein:
a first portion of the wavefront controller is that portion of a control area of the wavefront controller that is illuminated by the irradiation beam of light which is outside of the beam shape;
the wavefront controller sets the beam shape of the irradiation beam of light by applying a periodic spatial phase modulation pattern to the first portion of the wavefront controller; and
a period of the periodic spatial phase modulation pattern is substantially equal to half a wavelength of the irradiation light.

10. The method according to claim 9, wherein the periodic spatial phase modulation pattern is periodic along a first axis and is substantially constant along a second axis which is substantially orthogonal to the first axis.

11. The method according to claim 9, wherein the periodic spatial phase modulation pattern is substantially radially symmetric.

12. The method according to claim 11, wherein the beam shape is radially symmetric and a radial axis of symmetry for the periodic spatial phase modulation pattern is substantially coaxial with a center of the beam shape of the irradiation beam of light.

13. The method according to claim 9, wherein the wavelength of the irradiation light is selected from the group consisting of:
a peak wavelength of the irradiation light;
a mean wavelength of the irradiation light; and
a median wavelength of the irradiation light.

14. The method according to claim 1, wherein:
a first portion of the wavefront controller is that portion of a control area of the wavefront controller that is illuminated by the irradiation beam of light which is outside of the beam shape;
the wavefront controller sets the beam shape of the irradiation beam of light by applying a spatial phase modulation pattern to the first portion of the wavefront controller which causes a first aberration to be added to the irradiation beam of light that illuminates the first portion of the wavefront controller.

15. The method according to claim 14, wherein the first aberration includes one or more of:
a defocus aberration;
a conic aberration;
one directional tilt aberration;
two directional tilt aberration; and
a radial tilt aberration.

16. The method according to claim 1, wherein the measurement wavefront data includes information about aberrations introduced to the measurement light by the subject.

17. The method according to claim 1, wherein the beam shape is a circle, the wavefront controller is used to set a beam diameter of the irradiation light.

18. The method according to claim 1, wherein the first setting is a resolution setting and a size of the beam shape is calculated based on the resolution setting.

19. A non-transitory computer readable medium encoded with instructions for a one or more processors to control an adaptive optics fundus imaging apparatus with a wavefront controller that irradiates a subject's fundus with an irradiation light, and collects a measurement light from the subject, comprising:
receiving a first setting;
sending control data based on the first setting to the wavefront controller to adjust an irradiation wavefront so as to set a beam shape of the irradiation beam of light;
receiving measurement wavefront data that is representative of a wavefront of the measurement light collected from the subject; and
sending updated control data to the wavefront controller to readjust the irradiation wavefront based on the measurement wavefront data while at the same time also using the wavefront controller to control the beam shape of the irradiation beam of light based on the first setting.

20. An adaptive optics fundus imaging apparatus, comprising:
a wavefront controller, that changes a wavefront of irradiation light that is used to irradiate a subject's fundus;
a wavefront sensor for measuring a wavefront of measurement light collected from the subjects fundus that outputs measurement wavefront data that is representative of the wavefront of the measurement light;
one or more processors;
the one or more processors receive a first setting;
the one or more processors send control data based on the first setting to the wavefront controller to adjust an irradiation wavefront so as to set a beam shape of the irradiation beam of light;
the one or more processors receive the measurement wavefront data from the wavefront sensor; and
the one or more processors send updated control data to the wavefront controller to readjust the irradiation wavefront based on the measurement wavefront data while at the same time also using the wavefront controller to control the beam shape of the irradiation beam of light based on the first setting.
